(19) United States
(12) Patent Application Publication
(10) Pub. No.: US 2022/0381762 A1
(43) Pub. Date: Dec. 1, 2022

(54) NANOPORE FORMING METHOD AND NANOPORE MEASURING METHOD

(71) Applicant: Hitachi, Ltd., Tokyo (JP)

(72) Inventors: Sayaka TEZUKA, Tokyo (JP); Itaru YANAGI, Tokyo (JP)

(21) Appl. No.: 17/750,100

(22) Filed: May 20, 2022

(30) Foreign Application Priority Data
May 31, 2021 (JP) 2021-091022

(51) Int. Cl. G01N 33/487; G01N 27/447

(52) U.S. Cl. CPC G01N 33/48721; G01N 27/44791

(57) ABSTRACT

A nanopore forming method of the present disclosure includes: disposing a membrane between a first electrolyte solution and a second electrolyte solution; bringing a first electrode into contact with the first electrolyte solution and a second electrode into contact with the second electrolyte solution; and applying a first voltage between the first electrode and the second electrode to form a nanopore in the membrane. At least one of the first electrolyte solution or the second electrolyte solution contains a first substance that is an organic substance physically adsorbed or chemically adsorbed to the membrane to form a molecular layer.

### CROSS REFERENCE TO RELATED APPLICATIONS

The present application claims priority from Japanese patent application JP 2021-091022 filed on May 31, 2021, the entire content of which is hereby incorporated by reference into this application.

### BACKGROUND OF THE INVENTION

### 1. Field of the Invention

The present disclosure relates to a nanopore forming method and a nanopore measuring method.

### 2. Description of the Related Art

As a means for detecting molecules or particles present in an aqueous solution, a technique using a nanopore has been studied. In a nanopore device, a pore (nanopore) having the same size as that of a molecule or particle to be detected is provided in a membrane, and upper and lower chambers of the membrane are filled with an aqueous solution. Electrodes are provided in both the chambers so as to be in contact with the aqueous solution. At the time of measurement, an object to be detected which is an object to be measured is introduced into one side of the chambers, and a potential difference is applied between the electrodes to electrophorese the object to be detected, thereby causing the object to be detected to pass through the nanopore. At this time, by measuring the time change of an ion current (blockade current) flowing between both the electrodes, it is possible to detect the passage of the object to be detected and analyze the structural characteristics of the object to be detected.

Harold Kwok, et al., PloS ONE, Vol. 9, No. 3, e92880. (2014), Kyle Briggs, et al., Nanotechnology, Vol. 26, 084004 (2015), and Kyle Briggs, et al., Small, 10 (10): 2077-86 (2014) disclose nanopore forming methods using the dielectric breakdown phenomenon of a membrane. In these methods, first, each of upper and lower chambers sandwiching a SiNx membrane having no pore is filled with an aqueous solution. An electrode is immersed in the aqueous solution of each chamber, and a high voltage is continuously applied between both the electrodes. When the current between the electrodes rapidly increases (the dielectric breakdown of the membrane occurs), and reaches a predetermined cutoff current, a nanopore having a desired size is determined to be formed, and the application of a high voltage is stopped to form the nanopore.

Examples of the application of measurement using a nanopore include confirmation of the presence or absence, measurement of a size or shape, and determination of an active state, of an object to be detected present in an aqueous solution. Examples of the potential applications of the nanopore include, for example, medicine, biotechnology, life science, defense, public health, and agriculture. In order to enhance the accuracy of the measurement using the nanopore to promote commercial utilization, it is necessary that the nanopore measurement can be reproducibly performed for a long time.

In the nanopore measurement, an object to be measured is often adsorbed to the nanopore, whereby the stability of a signal obtained from the nanopore may be lost. Therefore, the development of a technique for suppressing the adsorption of an object to be measured in an aqueous solution to a nanopore has attracted attention. For example, Y M Nuwan D. Y. Bandara, et al. Nanotechnology 31 335707 (2020) describes that, when a nanopore is formed by a dielectric breakdown method, the surface of the nanopore is oxidized by adding NaClO to an opening solution to form the nanopore that is negatively charged in an aqueous solution. By the method of Y M Nuwan D. Y. Bandara, et al. Nanotechnology 31 335707 (2020), DNA having a negative charge can be suppressed from being adsorbed to the nanopore during measurement. Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) describe that non-specific adsorption of an object to be measured (DNA or protein) to a nanopore can be suppressed by coating an opened nanopore with a surfactant or a lipid bilayer membrane. In particular, Xiaoqing Li, et al., Appl. Phys. Lett. 109, 143105 (2016) describes that coating with a surfactant having no charge has an effect of suppressing the adsorption of both a positively charged object to be measured and a negatively charged object to be measured.

### SUMMARY OF THE INVENTION

When the object to be measured is adsorbed to the nanopore during measurement, a signal of the object to be detected passing through the nanopore later is buried in noise derived from an adsorbed matter, which may cause difficult detection. In some cases, the nanopore is clogged, and the object to be detected does not pass through the nanopore, whereby the measurement may be interrupted.

One of the causes of the adsorption of the object to be measured to the nanopore is interaction between the surface of the nanopore and the object to be measured. For example, in the surface of silicon nitride (SiN) frequently used in a semiconductor nanopore, a silanol group which is a kind of acidic surface group and a silaramine group which is a kind of basic surface group are mixed, and the surface of the nanopore is positively or negatively charged. Therefore, when molecules or particles having an opposite charge to that of the surface of the nanopore approach the nanopore, they may be drawn to the wall surface of the nanopore by the Coulomb force. The object to be measured drawn to the wall surface of the nanopore may be adsorbed to the wall surface of the nanopore by Coulomb interaction, hydrophobic interaction, hydrogen bonding, or van der Waals force or the like.

As described above, Y M Nuwan D. Y. Bandara, et al. Nanotechnology 31 335707 (2020) describes that the adsorption of DNA to the nanopore is suppressed by adding NaClO to the opening solution. However, it is considered that positively charged molecules are adsorbed to the nanopore opened by the method of Y M Nuwan D. Y. Bandara, et al. Nanotechnology 31 335707 (2020).

Examples of the method for forming the coating on the nanopore to suppress the adsorption of the object to be measured as in Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) include a method for placing, after forming an opening, a nanopore device in a dry state once, and then performing coating, and a method for performing coating by replacing a solution after forming an opening. In these methods, it takes about 10 minutes to 1 hour for coating, and it takes time and effort to perform a surface cleaning treatment that requires special equipment depending on the surface state of the nanopore device.

Therefore, the present disclosure provides a technique for coating a nanopore by a simple method.

In order to solve the above problems, a nanopore forming method of the present disclosure includes: disposing a membrane between a first electrolyte solution and a second electrolyte solution; bringing a first electrode into contact with the first electrolyte solution and bringing a second electrode into contact with the second electrolyte solution; and applying a first voltage between the first electrode and the second electrode to form a nanopore in the membrane, in which at least one of the first electrolyte solution or the second electrolyte solution contains a first substance that is an organic substance physically adsorbed or chemically adsorbed to the membrane to form a molecular layer.

Other features related to the present disclosure will be clear from the description and the accompanying drawings of the present specification. In addition, the aspects of the present disclosure are achieved and realized by elements, combinations of various elements, the following detailed description, and aspects of the appended claims. The description of the present specification is given only as a typical example, and does not limit the scope of claims or application examples of the present disclosure in any manner.

According to a technique of the present disclosure, a nanopore can be coated by a simple method. The problems, configurations, and effects other than those described above are apparent from the descriptions of the following embodiments.

### BRIEF DESCRIPTION OF THE DRAWINGS

FIG. 1 is a schematic diagram showing the configuration of a nanopore forming apparatus;

FIG. 2 is a schematic diagram showing another configuration of the nanopore forming apparatus;

FIG. 3 is a diagram showing a current waveform when a DNA sample is measured at an applied voltage of 100 mV;

FIG. 4 is a diagram showing a current waveform when adsorption occurs during measurement of the DNA sample;

FIG. 5 is a diagram showing a current waveform when adsorption occurs during measurement of a SA-DNA sample;

FIG. 6 is a diagram showing a current waveform when the adsorption of SA-DNA is eliminated by a change in an applied voltage;

FIG. 7 is a flowchart showing nanopore coating methods according to a conventional example and a first embodiment;

FIG. 8 is a schematic diagram showing a nanopore coating method according to Comparative Example 2;

FIG. 9 is a schematic diagram showing a nanopore coating method according to Example 1;

FIG. 10 is a schematic diagram showing a nanopore coating method according to Example 2;

FIG. 11 is a schematic diagram showing a nanopore coating method according to Comparative Example 3;

FIG. 12 is a graph showing average measurable times in measurement using nanopore devices of Comparative Examples 1 to 3 and Examples 1 to 4;

FIG. 13 is a diagram showing a current waveform when MBD2 is measured in Example 5;

FIG. 14 is a diagram showing current waveforms obtained by enlarging sections (i) to (vi) in FIG. 13;

FIG. 15 is a diagram showing a current waveform when MBD2 is measured in Comparative Example 4; and

FIG. 16 is a diagram showing current waveforms obtained by enlarging sections (i) to (viii) in FIG. 15.

### DESCRIPTION OF THE PREFERRED EMBODIMENTS

Hereinafter, embodiments of the present disclosure will be described in detail with reference to the drawings. In all the drawings for explaining the embodiments, those having the same functions are denoted by the same reference numerals, and the repeated description thereof is omitted as much as possible. A measurement method, and the structure and material of a device described in the embodiments are examples for embodying the idea of the present disclosure, and the present disclosure does not strictly specify the measurement principles, and the materials and dimensions of the device, and the like.

Specific voltage values, current values, and voltage application times described in the embodiments are examples for embodying the idea of the present disclosure, and the present disclosure does not strictly specify them. Specific types of coating agents, immersion times in the coating agents, and compositions of coating solutions described in the embodiments are examples for embodying the idea of the present disclosure, and are not examples for strictly defining chemical compositions and coating times. Specific types of objects to be measured, types of solutions, and concentrations thereof described in the embodiments are examples for embodying the idea of the present disclosure, and are not examples for strictly defining the chemical compositions.

In the present specification, a substance to be detected in a nanopore is referred to as an object to be detected (for example, nucleic acids, modified nucleic acids, and proteins and the like), and a substance contained in a solution with which the nanopore is in contact at the time of nanopore measuring is referred to as an object to be measured. The object to be measured includes both the object to be detected and other substance (for example, modified molecules of the object to be detected or impurities).

In the present specification, a substance that is physically adsorbed or chemically adsorbed to the surface of the nanopore to suppress the adsorption of the object to be measured to the nanopore is referred to as a coating agent, and a procedure of adsorbing the coating agent to the surface of the nanopore to form a molecular layer is referred to as coating.

### First Embodiment

### Configuration Example of Nanopore Forming Apparatus

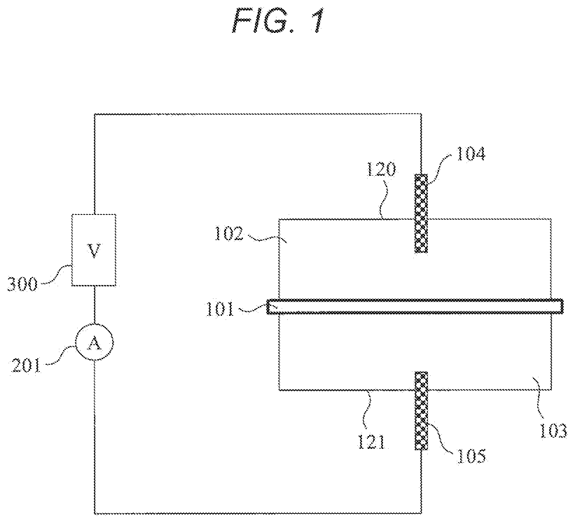

FIG. 1 is a simplified schematic diagram showing the configuration of a nanopore forming apparatus for a nanopore forming method. As shown in FIG. 1, the nanopore forming apparatus includes a membrane 101, electrodes 104 and 105, chambers 120 and 121, an ammeter 201, and a voltage source 300.

The chambers 120 and 121 are separated by the membrane 101. The material of the membrane 101 may be any material as long as a nanopore can be formed by dielectric breakdown, and for example, SiN, SiON, HfO₂, SiO₂, TiO₂, SiC, SiCN, Al₂O₃, HfAlOₓ, ZrAlOₓ, TaO₃, graphene, a carbon membrane, or a composite material containing these materials can be used. The thickness of the membrane 101 may be, for example, 1 μm or less. Specifically, as the membrane 101, for example, a silicon nitride membrane (SiN membrane) having a thickness of 3 to 30 nm can be used.

An aqueous solution 102 is accommodated in the chamber 120, and an aqueous solution 103 is accommodated in the chamber 121. The aqueous solutions 102 and 103 are used as a nanopore forming solution (opening solution) or a nanopore measurement solution. As an electrolyte in the aqueous solutions 102 and 103, for example, salts that perform ion conduction and react with an electrode, such as potassium salts (such as potassium chloride (KCl)), sodium salts (such as sodium chloride (NaCl)), lithium salts, rubidium salts, cesium salts, ammonium salts (such as ammonium chloride (NH₄Cl) and ammonium sulfate ((NH₄)₂SO₄)), and magnesium salts (such as magnesium chloride (MgCl₂) and magnesium sulfate (MgSO₄)) can be used. These electrolytes may be used alone or in combination of two or more thereof. Specifically, for example, a KCl aqueous solution can be used as the aqueous solutions 102 and 103. The concentration of the electrolyte may be, for example, 0.01 M or more and a saturation concentration or less. As the pH of the aqueous solutions 102 and 103, a value suitable for opening a nanopore can be appropriately selected. In particular, by setting the pH of the aqueous solutions 102 and 103 to 13.9 or less, damage to the nanopore forming apparatus can be prevented.

As a solvent for the solutions 102 and 103, it is possible to use a solvent which can stably disperse a biopolymer, does not dissolve an electrode, and does not inhibit electron transfer with the electrode. Specific examples of the solvent include water, alcohols (methanol, ethanol, and isopropanol and the like), acetic acid, acetone, acetonitrile, dimethylformamide, and dimethylsulfoxide. When a nucleic acid as the biopolymer is used as an object to be measured, water is typically used.

The electrode 104 is in contact with the aqueous solution 102 in the chamber 120, and the electrode 105 is in contact with the aqueous solution 103 in the chamber 121. The electrodes 104 and 105 are connected to the ammeter 201 and the voltage source 300. As the electrodes 104 and 105, for example, Ag/AgCl electrodes can be used. The electrodes 104 and 105 may be made of a material serving as a polarization electrode, and may be made of, for example, gold or platinum or the like. In that case, a substance capable of assisting an electron transfer reaction such as potassium ferricyanide or potassium ferrocyanide can be added to the measurement solution in order to secure a stable ionic current. Alternatively, substances capable of performing an electron transfer reaction, such as ferrocenes, can also be fixed on the surface of the polarization electrode.

The voltage source 300 applies any voltage between the electrodes 104 and 105. The ammeter 201 measures a current between the electrodes 104 and 105. Although not illustrated, the ammeter 201 and the voltage source 300 can be controlled using a control device such as a computer device or a dedicated control unit. The control device can cause a storage device (not illustrated) to record a current value measured by the ammeter 201 or cause the voltage source 300 to change an applied voltage on the basis of information of the measured current value.

The control device causes the ammeter 201 to measure a current flowing between the electrodes 104 and 105 while causing the voltage source 300 to continuously apply a constant voltage between the electrodes 104 and 105. The control device determines that a nanopore having a desired size is formed when the current value between the electrodes rises sharply due to the dielectric breakdown of the membrane 101 and reaches a preset predetermined threshold current, and stops the application of the voltage. Thereby, the nanopore is obtained.

The diameter of the nanopore can be changed by the object to be detected. For example, when a biopolymer or bead having a diameter of about 10 nm is analyzed, the diameter of the nanopore may be 100 nm or less. For example, the diameter of the nanopore used for analysis of ssDNA (single-stranded DNA) having a diameter of about 1.4 nm may be about 1.4 nm to 10 nm.

Examples of the object to be detected include: biopolymers such as nucleic acids, proteins, and polysaccharides; biomonomers such as amino acids, lipids, sugars, and nucleotides; derivatives of the biopolymers and biomonomers; nanoparticles, nanorods, and nanostructures of inorganic substances, metals, or organic substances; cells; organelles; and viruses. The size and length of the object to be detected are not particularly limited, but for example, the object to be detected may be a string-like substance having a diameter of about 0.1 nm to 500 nm or a substance having a diameter of 0.1 nm to 500 nm when approximated to a sphere.

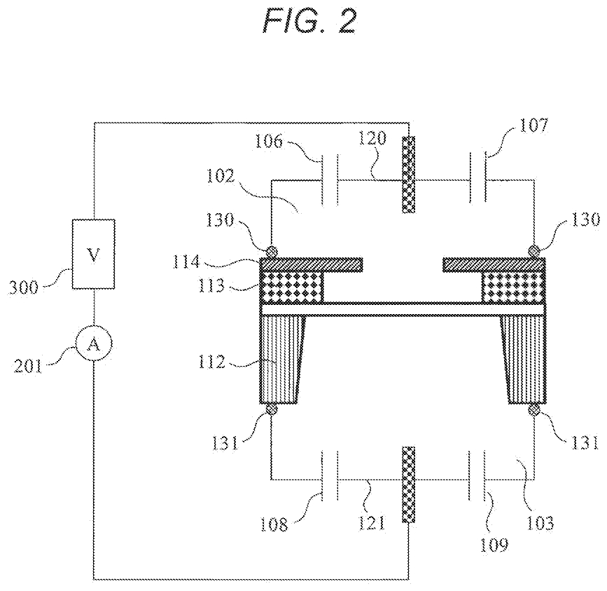

FIG. 2 is a schematic diagram showing another configuration of the nanopore forming apparatus. In the nanopore forming apparatus of FIG. 2, the membrane 101 is supported by a support substrate 112. As the material of the support substrate 112, for example, silicon (Si) can be used. A membrane 113 is laminated on the upper surface of the membrane 101, and a membrane 114 is laminated on the membrane 113. As the material of the membrane 113, for example, silicon oxide (SiO₂) or silicon (Si) can be used. As the material of the membrane 114, for example, silicon nitride (SiNₓ) can be used.

The chamber 120 is provided with a solution inlet 106 and a solution outlet 107, and the chamber 121 is provided with a solution inlet 108 and a solution outlet 109. Furthermore, a sealant 130 is disposed between the membrane 101 and the chamber 120, and a sealant 131 is disposed between the support substrate 112 and the chamber 121. The sealants 130 and 134 are, for example, O-rings, and respectively prevent the leakage of aqueous solutions in the chambers 120 and 121.

Hereinafter, in the present specification, for simplification of illustration, a simplified diagram as in FIG. 1 in which the solution inlets 106 and 108, the solution outlets 107 and 109, the support substrate 112, and the sealants 130 and 131 are omitted is used. In the present specification, the “nanopore device” refers to a component other than the ammeter 201 and the voltage source 300 of the nanopore forming apparatus, that is, a portion including the membrane 101, the support substrate 112, the membranes 113 and 114, the electrodes 104 and 105, and the chambers 120 and 121. The nanopore device is connected to the ammeter 201 and the voltage source 300 by wirings to constitute the nanopore forming apparatus.

### <Method for Producing Nanopore Device>

A method for producing a nanopore device and a method for forming a nanopore are known, and are described in, for example, Itaru Yanagi et al., Sci. Rep. 9: 13143 (2019). A general nanopore device can be performed, for example, by the following procedure.

First, Si₃N₄, SiO₂, and Si₃N₄ are respectively deposited to thicknesses of 14 nm, 260 nm, and 90 nm on the surface of an 8-inch Si wafer with a thickness of 725 μm, and Si₃N₄ is deposited to a thickness of 90 nm on the back surface. Next, reactive ion etching is applied to an area of 600 nm square of Si₃N₄ on the uppermost part of the front surface, and to an area of 1038 μm square of Si₃N₄ on the back surface. Furthermore, the Si substrate exposed through etching of the back surface is etched with tetramethylammonium hydroxide (TMAH). Next, the SiO₂ layer exposed in an area of 600 nm square is removed with a KOH aqueous solution (33% by weight, about 70° C., about 30 minutes). As a result, a thin membrane device in which a Si₃N₄ thin membrane (membrane) having a membrane thickness of 14 nm is exposed is obtained. At this stage, a nanopore is not provided in the thin membrane yet. The thin membrane device is set in the nanopore forming apparatus so as to separate the two upper and lower chambers (chambers 120 and 121) with the thin membrane device produced as described above, to obtain the nanopore device.

### <Nanopore Forming Method>

A nanopore can be formed in the thin membrane by applying a DC voltage, for example, according to the following procedure. First, each chamber is filled with an opening solution containing 1 M KCl and 1 mM Tris-10 mM EDTA, and having a pH of 12.7, and Ag/AgCl electrodes (electrodes 104 and 105) are introduced into the chamber. Application of a voltage for forming a nanopore and measurement of an ion current that flows through the formed nanopore are conducted between the Ag/AgCl electrodes.

Here, the lower chamber (chamber 121) is referred to as a cis chamber, and the upper chamber (chamber 120) is referred to as a trans chamber. A voltage Vcis applied to the cis chamber side electrode is set to 0 V, and a voltage Vtrans applied to the trans chamber side electrode is set to −11 V (hereinafter, a case where the voltage Vcis is fixed to 0 V and the voltage Vtrans is changed will be described). The value of a current flowing when the DC voltage is applied can be read using a current amplifier (4156B PRECISION SEMICONDUCTOR ANALYZER manufactured by Agilent Technologies, Inc.). The processes of applying a voltage for forming a nanopore and reading an ion current are controlled using a program produced by the inventors (Excel VBA, Visual Basic for Applications).

The diameter of the nanopore can be estimated from the ion current value. By selecting the condition (threshold current) of the current value to be acquired according to the diameter of the nanopore formed in the thin membrane when the DC voltage is applied, the nanopore having a target diameter can be obtained. In the above example, the applied DC voltage is −11 V, and by setting the condition of the threshold current to 450 to 650 nA, the nanopore having a diameter of 9 to 11 nm can be formed.

### <Nanopore Measuring Method>

An example of measurement using the nanopore formed by the above procedure will be described. First, a measurement solution (hereinafter, referred to as a DNA sample) obtained by mixing 3 nM of DNA of 415 bp (object to be detected) with an aqueous solution containing 0.1 M KCl and 1 mM Tris-10 mM EDTA-1 mM (tris-ethylenediaminetetraacetic acid solution), and having a pH of 8.0 is introduced into the cis chamber. Next, an electrode (electrode 105) in contact with the cis chamber (chamber 121) is connected to a negative electrode of a power supply. An electrode (electrode 104) in contact with the trans chamber (chamber 120) is connected to a positive electrode of the power supply. A DC voltage is applied in the range of 100 mV to 400 mV. The time change of a current when DNA passes through the nanopore is acquired by an ultra-low noise patch clamp amplifier (Axopatch 200B). A DNA sample was measured for up to 24 minutes.

Subsequently, 500 nM of 415 bp DNA having a terminal modified with biotin and 5 μM of streptavidin are mixed to form a complex of streptavidin and DNA (hereinafter, referred to as SA-DNA). A measurement solution (hereinafter, referred to as SA-DNA sample) obtained by diluting SA-DNA (object to be detected) 500 times with an aqueous solution containing 0.1 M KCl and 1 mM Tris-10 mM EDTA, and having a pH of 8.0 is introduced into the cis chamber. The SA-DNA sample is measured in the same manner as the DNA sample for up to 24 minutes. The isoelectric point of streptavidin used in the present specification is a pH of 6.5 to 7.5, whereby the streptavidin is considered to be negatively charged in a measurement solution having a pH of 8.0.

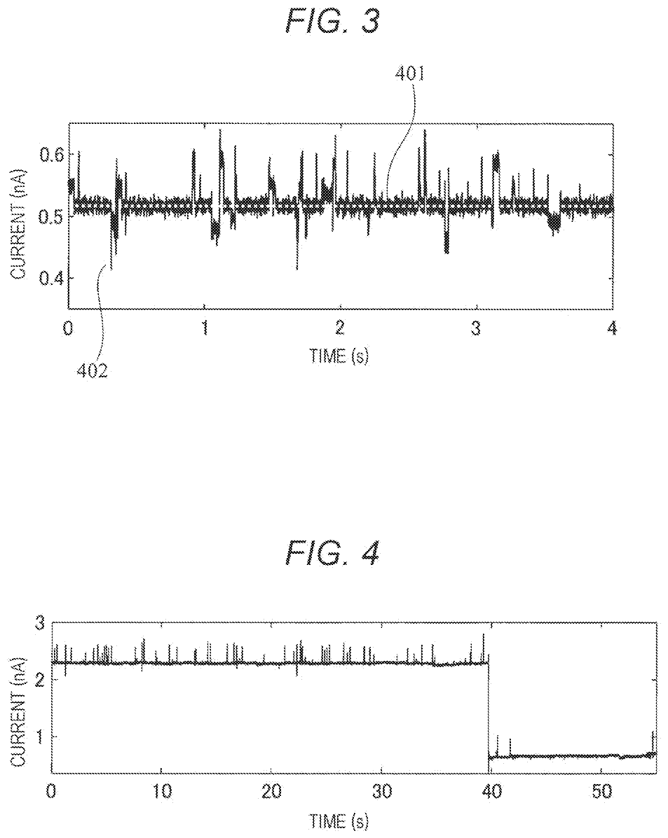

FIG. 3 is a diagram showing a current waveform when a DNA sample is measured at an applied voltage of 100 mV. In FIG. 3, a horizontal axis represents a time (second), and a vertical axis represents a current value (nA). As shown in FIG. 3, a base current 401 and a passage current waveform 402 can be clearly distinguished from each other from the current waveform. The base current 401 is a current flowing through the nanopore when the object to be measured does not pass through the nanopore. The passage current waveform 402 includes a waveform in which the nanopore is partially blocked when the object to be measured passes through the nanopore to decrease a conductivity, and a waveform in which DNA draws ions into the nanopore to increase a conductivity. Usually, a time taken for the object to be measured to pass through the nanopore is 1 second or less, and varies depending on conditions such as the length of the object to be measured or the applied voltage.

As a method for detecting the object to be detected, it is also possible to measure optical signals such as absorption, reflection, and fluorescence characteristics of light emitted to the vicinity of the nanopore, instead of the method for measuring the blockade current as described above.

### <Regarding Adsorption of Object to be Measured to Nanopore>

When the nanopore is not coated, a phenomenon often occurs, in which an object to be measured is adsorbed to the nanopore at the time of nanopore measuring, whereby a clear passage waveform is not observed.

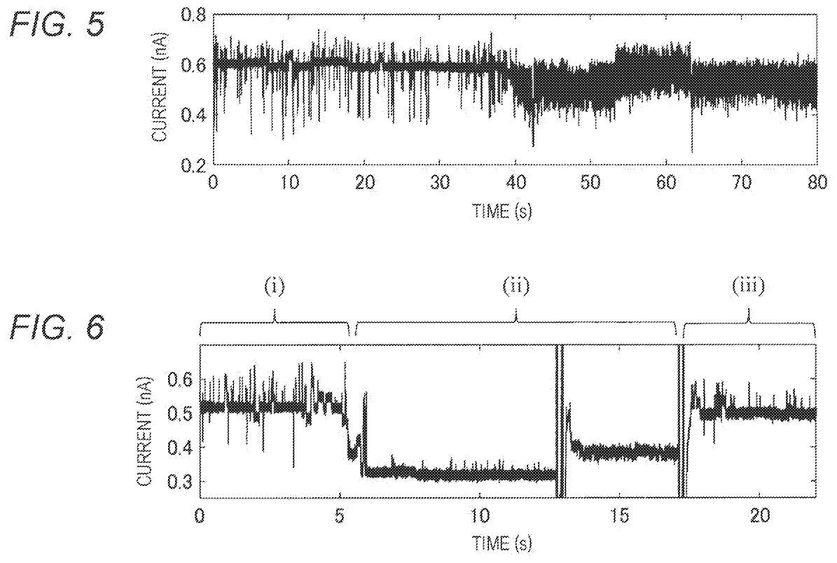

FIG. 4 is a diagram showing a current waveform when adsorption occurs during measurement of a DNA sample. FIG. 5 is a diagram showing a current waveform when adsorption occurs during measurement of a SA-DNA sample. FIGS. 4 and 5 merely show typical examples of adsorption, and do not show a waveform at the time of adsorption specific to the object to be measured.

As shown in FIG. 4, a DNA passage waveform is observed until t=40 seconds, but a base current decreases from about 2.3 nA to about 0.5 nA at t=40 seconds. This is considered to be because DNA (object to be measured) is adsorbed to the nanopore, whereby a part of the nanopore is blocked.

As shown in FIG. 5, it can be seen that the SA-DNA continuously passes through the nanopore until t=38 seconds, but after t=38 seconds, the noise of the base current increases, whereby the passage of the SA-DNA and the noise cannot be distinguished from each other. This noise is considered to reflect a state where SA-DNA (object to be measured) is adsorbed to the nanopore and vigorously vibrates. As described above, when the object to be measured is adsorbed to the nanopore, (1) a phenomenon in which the base current value significantly decreases over 1 second or more and/or (2) a phenomenon in which the noise of the base current increases are observed.

A method for eliminating the adsorption of the object to be measured occurring during the nanopore measurement is a method for changing an applied voltage, more specifically, a method for performing ZAP. The ZAP is a method for changing the applied voltage within a range of, for example, ±1.3 V (applying a pulse voltage). The adsorption of the object to be measured includes adsorption that is eliminated by changing the applied voltage and adsorption that is not eliminated even if the applied voltage is changed. Here, the adsorption eliminated by the change in the applied voltage is referred to as reversible adsorption. Meanwhile, adsorption that is not eliminated even when ZAP is performed 20 times or more is referred to as irreversible adsorption. The ZAP can be performed, for example, for 0.5 milliseconds to 1 second per one time.

FIG. 6 is a diagram showing a current waveform when SA-DNA is adsorbed to the nanopore and the adsorption of SA-DNA is eliminated by a change in an applied voltage. In FIG. 6, the current waveform clearly changes from 0 seconds to 10 seconds, whereby a condition in which SA-DNA as an object to be detected passes through the nanopore can be confirmed. However, at time t=5 seconds, the adsorption of an object to be measured (SA-DNA or other substances contained in the sample) occurs, and the baseline current decreases from 0.5 nA to 0.3 nA. Therefore, ZAP is performed at t=13 seconds and t=17 seconds to eliminate the adsorption.

Whether the adsorption of the object to be measured to the nanopore becomes reversible or irreversible depends on the strength of the adsorption. In the case of the reversible adsorption, the measurement can be resumed as soon as the adsorption is eliminated, which has no problem. Meanwhile, when the irreversible adsorption occurs, the measurement must be interrupted. Here, the total measurement time of each sample (DNA sample and SA-DNA sample) until the irreversible adsorption occurs is referred to as a measurable time. When the irreversible adsorption does not occur during the measurement, the measurable time is the sum of the maximum measurement times of the samples.

In the examples of the DNA sample and the SA-DNA sample described above, the measurable time is 48 minutes, which is the sum of the maximum measurement time (24 minutes) of the DNA sample and the maximum measurement time (24 minutes) of the SA-DNA sample. Sections (i) and (iii) in FIG. 6 are included in the measurement time of the SA-DNA sample. A section from the clogging of the nanopore to the recovering of the nanopore (section (ii) in FIG. 6) may not be included in the measurement time, or may be included in the measurement time if the section is short (for example, 10 seconds or less).

### <Nanopore Coating Method>

A method for suppressing the adsorption of an object to be measured to a nanopore during nanopore measurement is coating of the nanopore. Hereinafter, nanopore coating methods according to a conventional example and the present embodiment will be described.

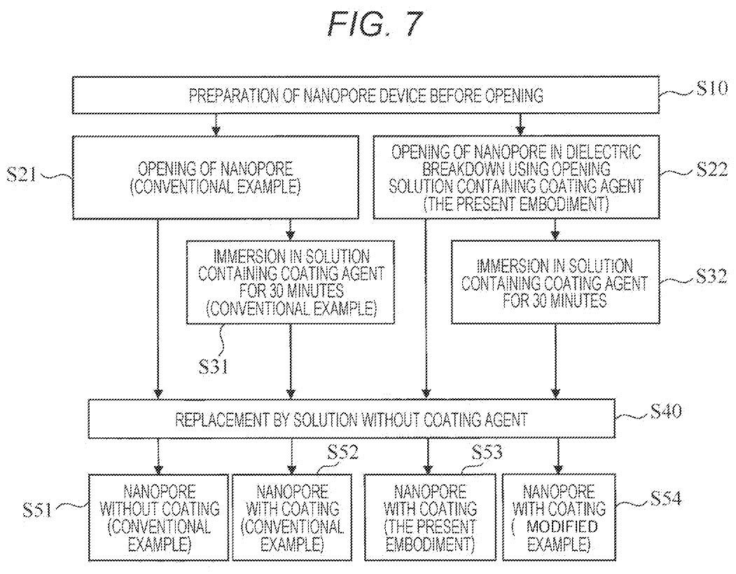

FIG. 7 is a flowchart showing nanopore coating methods according to the conventional example and the present embodiment.

### Nanopore Coating Method According to First Conventional Example

As a first conventional example, a method for coating a nanopore formed by a dielectric breakdown method with a surfactant as in Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) will be described. Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) describe that a nanopore formed by a transmission electron microscope (TEM) or the like is immersed in an aqueous solution containing 0.01 to 0.1% by weight of polyoxyethylene (20) sorbitan monolaurate (Tween (registered trademark) 20), which is a kind of surfactant, for 10 minutes to 1 hour to perform coating. The coating methods of Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) can be similarly performed on a nanopore formed by a dielectric breakdown method.

In step S10, a user prepares a nanopore device (FIG. 1 or 2) before opening a nanopore, and sets the nanopore device in a nanopore forming apparatus.

In step S21, the user introduces a known opening solution into each chamber. Next, a control device of the nanopore forming apparatus applies a voltage to electrodes of each chamber to form a nanopore by the dielectric breakdown of a membrane. As the pH of the opening solution, a value suitable for opening the nanopore can be appropriately selected. For example, the pH of the opening solution can be set to 12.7.

In step S31, the user replaces the opening solution as a liquid in each chamber by a solution with a coating agent, and leaves the solution (immerses the nanopore) for 30 minutes, for example. Xiaoqing Li, et al., Appl. Phys. Lett. 109, 143105 (2016), Jared Houghtaling, et al., ACS Nano 13, 5, 5231-5242 (2019), and Rui Hu, et al., Sci Rep 6, 20776 (2016) describe that polyoxyethylene (20) sorbitan monolaurate (Tween 20) as a surfactant is used as the coating agent.

In step S40, the user replaces the solution with a coating agent as the liquid in each chamber by a solution containing no coating agent (for example, a cleaning liquid or a measurement solution or the like).

In step S52, the user can acquire a nanopore device with coating according to the first conventional example.

A method for forming a nanopore without coating is the same as that in the above-described first conventional example except that step S31 is not performed.

### Nanopore Coating Method According to the Present Embodiment

A nanopore coating method according to the present embodiment is different from that in the first conventional example in that an opening solution with a coating agent is used at the time of forming a nanopore. The coating method of the present embodiment is as follows.

Step S10 is similar to that in the first conventional example. After step S10, the process proceeds to step S22.

In step S22, the user introduces an opening solution (a first electrolyte solution and a second electrolyte solution) with a coating agent into each chamber. The coating agent will be described in detail later. A control device of a nanopore forming apparatus applies a voltage to electrodes to form a nanopore by the dielectric breakdown of a membrane. As the pH of the opening solution with a coating agent, a value suitable for opening a nanopore can be appropriately selected. For example, the pH of the opening solution with a coating agent can be set to 12.7. In the present step, an opening solution with a coating agent may be introduced into one chamber, and an opening solution containing no coating agent may be introduced into the other chamber.

After step S22, step S40 is performed in the same manner as described above. Thereafter, in step S53, the user can acquire a nanopore device with coating according to the present embodiment. Thereafter, the opening solution as the solution in the chamber is replaced by a measurement solution, whereby the nanopore measurement of the object to be measured can be performed.

### Method for Forming Nanopore with Coating According to Modified Example of the Present Embodiment

After a nanopore is formed using an opening solution with a coating agent, a solution in a chamber may be replaced by a coating solution (third electrolyte solution) having a property different from that of the opening solution to immerse the nanopore, thereby performing further coating. The coating method of the present modified example is as follows.

Steps S10 and S22 are as described above. After step S22, the process proceeds to step S32.

In step S32, the user replaces the opening solution as a liquid in each chamber by a coating solution, and leaves the solution (immerses the nanopore) for 30 minutes, for example.

As the pH of the coating solution, a value that increases the affinity between the coating agent and the surface of the nanopore can be appropriately selected. For example, when a positively charged coating agent is used, an aqueous solution having a pH at which a nanopore is negatively charged can be used, and when a negatively charged molecule is used as a coating agent, an aqueous solution having a low pH at which a nanopore is positively charged can be used.

The immersion time of the nanopore in the coating solution is not limited to 30 minutes, and can be set according to conditions such as the type of the coating agent or the material of the nanopore. For example, the immersion time can be set to 5 minutes or more and 240 hours or less. The coating agent in the opening solution used in step S22 (at the time of opening the nanopore) and the coating agent in the coating solution used in step S32 (after opening) may be the same or different.

After step S32, step S40 is performed in the same manner as described above. Thereafter, in step 354, the user can acquire a nanopore device with coating according to the modified example.

### (Regarding Coating Agent)

The coating agent is an organic substance capable of being physically adsorbed or chemically adsorbed to a membrane on which a nanopore is formed, to form a molecular layer. The coating agent may be a substance that interacts with the surface of the nanopore, more specifically, a substance having a structure capable of being adsorbed to the surface of the nanopore. Alternatively, the coating agent may be a substance having a hydrophilic structure.

Specific examples of the coating agent include surfactants (nonionic surfactants, anionic surfactants, cationic surfactants, or amphoteric surfactants), biomolecules (such as peptides or lipids), and polymers other than biomolecules.

Examples of the nonionic surfactant include polyoxyethylene sorbitan fatty acid ester (trade name: Tween (registered trademark)), polyoxyethylene alkylphenyl ether (trade name: Triton (registered trademark)), polyoxyethylene alkyl ether (trade name: Brij), alkylpolyglycosides such as n-dodecyl-Q-D-maltoside (DDM), and digitonin.

Examples of the anionic surfactant include sulfate esters such as sodium dodecyl sulfate (SDS), cholates, and sarcosyl.

Examples of the cationic surfactant include alkyltrimethylammonium bromides such as cetyltrimethylammonium bromide (CTAB).

Examples of the amphoteric surfactant include 3-[(3-cholamidopropyl)dimethylammonio]-1-propanesulfonate (CHAPS) and 3-[(3-cholamidopropyl)dimethylammonio]-2-hydroxy-1-propanesulfonate (CHAPSO).

Examples of the peptide which can be used include a substance having L-3,4-dihydroxyphenylalanine (DOPA) and a hydrophilic amino acid in the sequence, which is known to be adsorbed to a solid nanopore, or peptides that are specifically adsorbed to a solid interface and modified peptides thereof as described in Yoichi Kumada, et al., J Biotechnol. August 20; 184: 103-10 (2014).

Examples of the polymer other than the biomolecule include a molecule having a hydrophobic moiety at one end portion and a hydrophilic moiety at another end portion in a molecular structure, or an amphiphilic molecule having a hydrophobic moiety at one portion and a hydrophilic moiety at another portion when having a three-dimensional structure. Specific examples thereof include 2-methacryloyloxyethyl phosphorylcholine (MPC) and a copolymer composed of 2-aminoethyl vinyl ether and isobutyl vinyl ether.

When the object to be measured is positively or negatively charged, a coating agent (CTAB, SDS, and peptide having an acid amino acid/basic amino acid sequence, and the like) having a portion charged as in the charge of the object to be measured is used, whereby a repulsive force between the coating agent and the object to be measured acts. This can provide a high adsorption suppression effect.

As the coating agent, the above-described substances may be used alone or in combination of two or more thereof. The concentration of the coating agent may be, for example, 1 pM or more and a saturated concentration or less, or 0.0001% by weight or more and a saturated concentration or less.

The nanopore forming apparatus before forming the nanopore described above may be provided to a user in a state where an opening solution containing a coating agent is filled in each chamber. Alternatively, a nanopore forming apparatus with each empty chamber before forming the nanopore, and an opening solution containing a coating agent may be provided to a user as a nanopore forming kit. The coating agent may be provided as a concentrated solution for dilution with a suitable solvent during use, or may be in a solid state for reconfiguration with a suitable solvent during use (for example, powder and the like).

### Summary of First Embodiment

As described above, the nanopore forming method according to the present embodiment has been made by newly finding that the coating agent (organic molecule) can be adsorbed to the surface of the nanopore by adding the coating agent to the solution when the nanopore is formed by dielectric breakdown. According to the method of the present embodiment, even if the nanopore is not immersed in the coating solution after the formation of the nanopore, the nanopore can be easily coated simultaneously with the opening of the nanopore, that is, in a short time. In contrast, the nanopore coating method of Xiaoqing Li, et al., Appl. Phys. Lett. 109, 143105 (2016) makes it necessary to immerse the nanopore after forming in a surfactant solution for a predetermined time.

The method of the present embodiment is a method for coating the surface of the nanopore with the organic molecule by physical adsorption or chemical adsorption, whereby the method has an effect of suppressing adsorption not only for negatively charged molecules but also for positively charged molecules. In contrast, in Y M Nuwan D. Y. Bandara, et al. Nanotechnology 31 335707 (2020), the surface of the nanopore is oxidized and negatively charged by adding NaClO into the aqueous solution when the nanopore is formed by dielectric breakdown, whereby the adsorption of only the negatively charged object to be measured (DNA) to the nanopore can be suppressed.

Furthermore, according to the method of the present embodiment, the coating agent (organic molecule) is adsorbed immediately after the nanopore is opened, that is, in a state where the substance is not adsorbed to the surface of the nanopore, whereby the adsorption of substances other than the coating agent is suppressed. As a result, according to the method of the present embodiment, a high adsorption suppression effect can be achieved. As described above, when the organic molecule is added to the aqueous solution at the time of opening, the coating of the organic molecule on the nanopore is promoted, so that a higher effect of suppressing clogging is obtained as compared with the case of performing coating by immersing the nanopore device in the organic molecule solution after the formation of the nanopore. This phenomenon is a phenomenon newly found in the present disclosure, and cannot be easily inferred from the above-described known examples.

### EXAMPLES

Hereinafter, Examples of the technique of the present disclosure will be described.

### 1. Production of Nanopore Device

### Comparative Example 1: Without Coating

As Comparative Example 1, a nanopore device without coating was produced. Specifically, first, a nanopore device before forming the nanopore was prepared in the same manner as the method described in the section <Method for Producing Nanopore Device> described above.

Next, similarly to the method described in the section <Nanopore Forming Method> described above, the nanopore device was set in a nanopore forming apparatus, and a nanopore was formed by the dielectric breakdown of a membrane using an opening solution (pH: 12.7) containing 1 M KCl and 1 mM Tris-10 mM EDTA. Thereby, the nanopore device according to Comparative Example 1 was obtained.

### Comparative Example 2: Coating after Opening

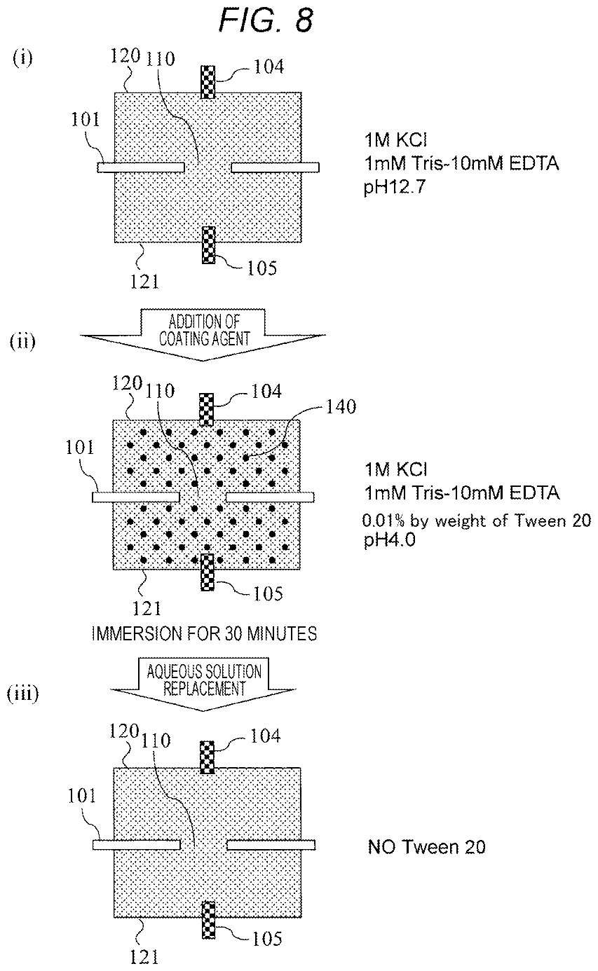

FIG. 8 is a schematic diagram showing a nanopore coating method according to Comparative Example 2. As shown in FIG. 8, first, a nanopore device with a nanopore 110 formed was produced in the same manner as in Comparative Example 1 (i). Thereafter, each chamber was filled with an aqueous solution (pH: 4.0) containing 1 M KCl, 1 mM Tris-10 mM EDTA, and 0.01% by weight of Tween 20, and immersed for 30 minutes to perform coating (ii). The solution in each chamber was then replaced by a solution containing no Tween 20 (iii).

### Example 1: Coating During Opening

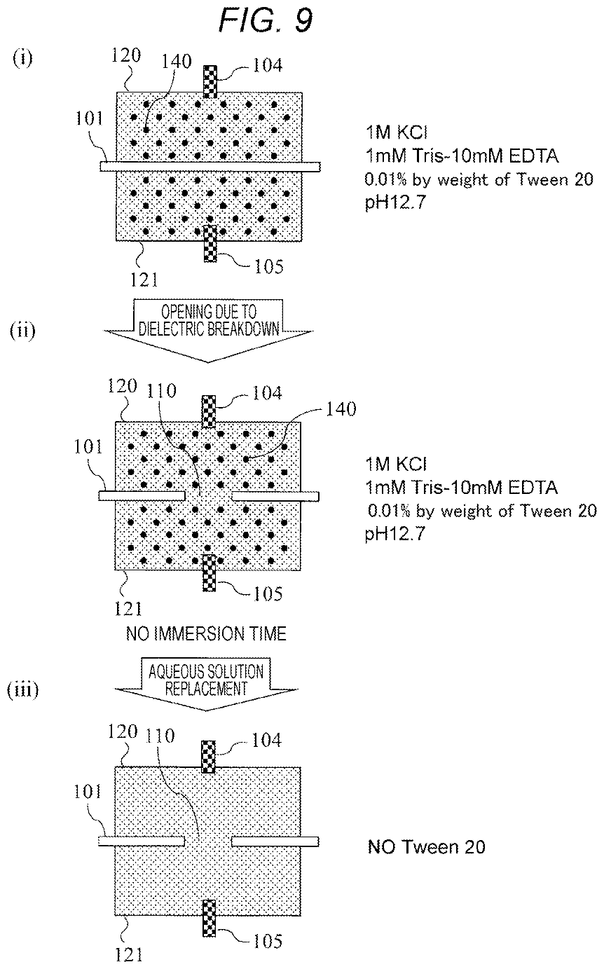

FIG. 9 is a schematic diagram showing a nanopore coating method according to Example 1. As shown in FIG. 9, first, a nanopore device before forming the nanopore was prepared in the same manner as the method described in the section <Method for Producing Nanopore Device> described above. An aqueous solution (pH: 12.7) containing 1 M KCl, 1 mM Tris-10 mM EDTA, and 0.01% by weight of Tween 20 was introduced into each chamber as an opening solution (i).

Next, −11 V was applied to an electrode 104, and 0 V was applied to an electrode 105 to form the nanopore 110 by dielectric breakdown (ii). Immediately after the opening of the nanopore, the solution in each chamber was replaced by a solution containing no Tween 20 (iii).

### Example 2: Coating During and after Opening

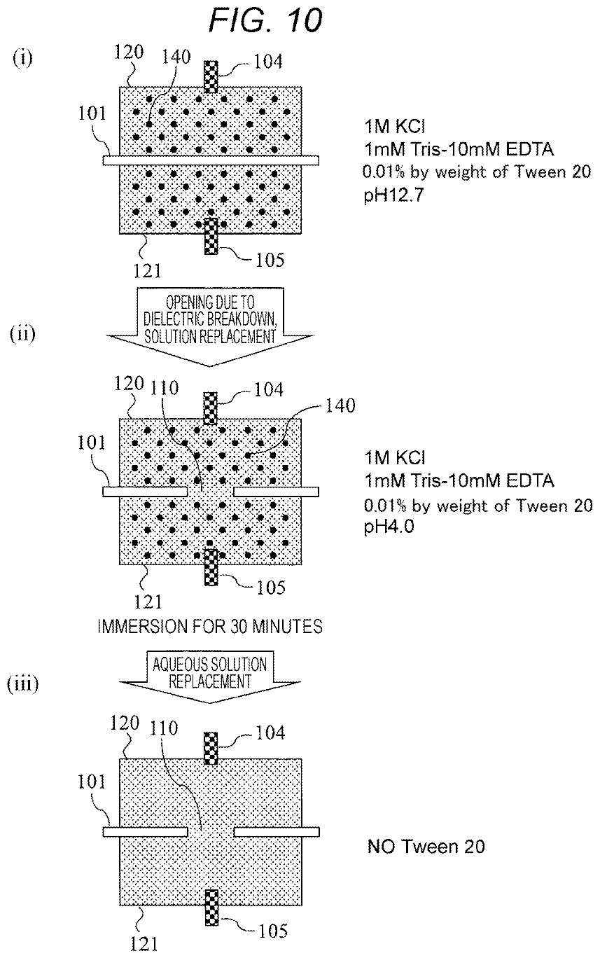

FIG. 10 is a schematic diagram showing a nanopore coating method according to Example 2. As shown in FIG. 9, first, in the same manner as in Example 1, a nanopore device before forming the nanopore was prepared, and an aqueous solution (pH: 12.7) containing 1 M KCl, 1 mM Tris-10 mM EDTA, and 0.01% by weight of Tween 20 was introduced into each chamber as an opening solution (i).

Next, −11 V was applied to an electrode 104, 0 V was applied to an electrode 105 to form a nanopore 110 by dielectric breakdown. The solution in the chamber was then replaced by a coating solution (an aqueous solution (pH: 4.0) containing 1 M KCl, 1 mM Tris-10 mM EDTA, and 0.01% by weight of Tween 20), and the nanopore 110 was immersed in the coating solution for 30 minutes (ii).

After the immersion for 30 minutes, the solution in each chamber was replaced by a solution containing no Tween 20 (iii).

### Comparative Example 3: Coating During Measurement

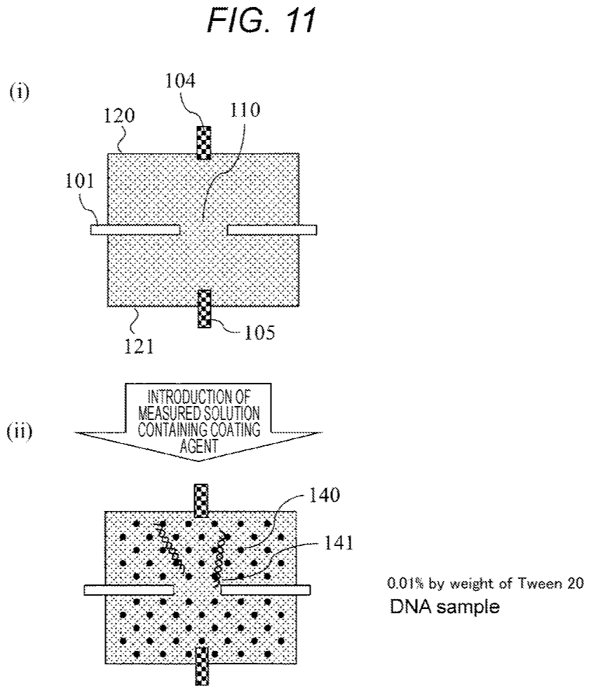

FIG. 11 is a schematic diagram showing a nanopore coating method according to Comparative Example 3. As shown in FIG. 11, first, a nanopore device with a nanopore 110 formed was produced in the same manner as in Comparative Example 1 (i). Thereafter, a measurement solution containing 0.01% by weight of Tween 20 as a coating agent 140 and 415 bp DNA as an object to be detected 141 was introduced into each of chambers 120 and 121. A voltage was applied between electrodes 104 and 105 to perform the coating of the nanopore 110 simultaneously with the measurement of a DNA blockade current (ii).

### Example 3: Change in Concentration of Coating Agent

A nanopore device according to Example 3 was obtained in the same manner as in Example 1 except that the concentration of Tween 20 as an opening solution was changed from 0.01% to 0.1%.

### Example 4: Change in Type of Coating Agent

A nanopore device according to Example 4 was obtained in the same manner as in Example 2 except that a coating agent in an opening solution and a coating agent in a coating solution were changed from Tween 20 to sodium dodecyl sulfate (SDS).

### 2. Evaluation of Measurable Time

Six nanopore devices according to each of Comparative Examples 1 to 3 and Examples 1 and 2 were produced. Three nanopore devices according to each of Examples 3 and 4 were produced. A DNA sample and a SA-DNA sample were introduced into each nanopore device, and an ion current was measured. A measurable time was recorded for each nanopore device, and an average measurable time was calculated. The effect of suppressing the adsorption of the coating in each of Comparative Examples and Examples is evaluated by the measurable time.

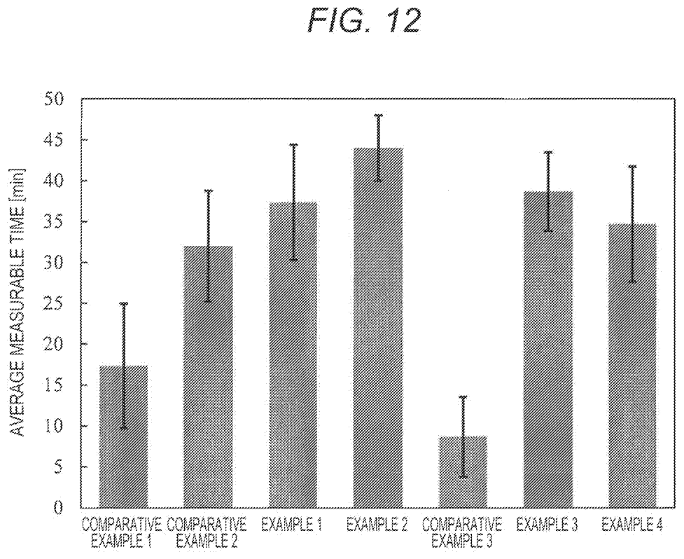

FIG. 12 is a graph showing average measurable times in measurement using nanopore devices of Comparative Examples 1 to 3 and Examples 1 to 4.

As shown in FIG. 12, it could be confirmed that the average measurable time when coating is performed (Examples 1 to 4 and Comparative Example 2) is longer than that in the case without coating (Comparative Example 1), to provide a suppressing effect on the adsorption of the object to be measured. The average measurable time in Example 1 was longer than the average measurable time in Comparative Example 2, whereby it was confirmed that by performing coating simultaneously at the time of the opening of the nanopore, a higher adsorption suppression effect than in the case of performing coating after the opening of the nanopore is provided. As is apparent from the above, the coating method according to the technique of the present disclosure does not require an immersion time, whereby the coating method can be performed in a short time, and has a higher adsorption suppression effect than that of the conventional method.

It can be seen that the average measurable time in Example 2 is longer than the average measurable time in Comparative Example 1 and the average measurable time in Example 1. From this, it was confirmed that the adsorption suppression effect can be improved by performing coating at the time of the opening of the nanopore and after the opening of the nanopore as in Example 2.

As in Comparative Example 3, the average measurable time when the measurement was performed in a state where the coating agent was directly mixed with the DNA sample without performing the coating before the measurement (coating at the time of the measurement) was shorter than that when the coating was not performed (Comparative Example 1). Since irreversible adsorption occurred within 24 minutes in any of devices of Comparative Example 3, the measurement of the SA-DNA sample could not be performed. From this experimental result, it was suggested that the coating agent released in the measurement solution has no suppressing effect on the adsorption of the object to be measured.

From the above results, it was found that the effect of suppressing the adsorption of the object to be measured to the nanopore is higher in the order of coating at the time of opening and coating after opening >coating at the time of opening >coating after opening >coating at the time of measuring. The adsorption suppression effect is considered to depend on the coverage of the coating agent and the persistence of the coating. It is considered that the coverage of the coating agent varies depending on the degree of activity of the surface at the start of coating and the time of coating, and the persistence of the coating agent varies depending on the adsorption property of the coating agent to the nanopore and the strength of a bonding force. Here, the reason why the effect of coating at the time of opening is higher than that of coating after opening is considered to be that the surface of the nanopore formed by dielectric breakdown has the highest degree of activity and high reactivity immediately after opening. That is, it is considered that when a molecule that is apt to be adsorbed to the surface of the nanopore is present in the opening solution, the nanopore can be coated with a high density as compared with the case where the molecule is introduced after opening. The degree of activity of the surface of the nanopore after elapse of time after opening decreases due to the influence of contaminants and the like in the air. It is also conceivable that the coating molecules are indirectly adhered onto the contaminants. In this case, it is considered that the fixing of the coating agent is weakened, causing an influence that the duration of the effect of the coating is shorter than that of the coating at the time of opening.

It is found that the average measurable time when the concentration of the coating agent is 0.1% by weight in Example 3 is longer than that when the concentration is 0.015 by weight in Example 1. Only the case where the concentration of the coating agent is 0.01 to 0.1% by weight has been described, but it is considered that the adsorption suppression effect can be obtained even when the concentration is changed to a concentration outside this range (for example, 1 pM or more and a saturated concentration or less, or 0.0001% by weight or more and a saturated concentration or less).

It could be confirmed that the average measurable time even when sodium dodecyl sulfate (SDS) is used as the coating agent as in Example 4 is longer than that in Comparative Example 1, to provide an effect of suppressing the adsorption of the object to be measured to the nanopore.

### 3. Change of Object to be Measured

### Example 5: Measurement of Protein

Coating of a nanopore device was performed in the same manner as in Example 2. Methyl-CpG-binding domain 2 (MBD2), which was a positively charged protein, was prepared as an object to be measured. MBD2 is a protein that specifically recognizes and binds to methylated DNA, and the theoretical value of the isoelectric point of MBD2 is a pH of 10.06. Thereafter, a solution (pH: 8.7) containing 0.1 M KCl, 1 mM Tris-10 mM EDTA, and 100 nM MBD2 was prepared as a measurement solution, and the measurement solution was introduced into a cis chamber. In this measurement solution, MBD2 is considered to be positively charged. Next, a voltage was applied in the range of 100 to 300 mV between Ag/AgCl electrodes, and an ion current was measured.

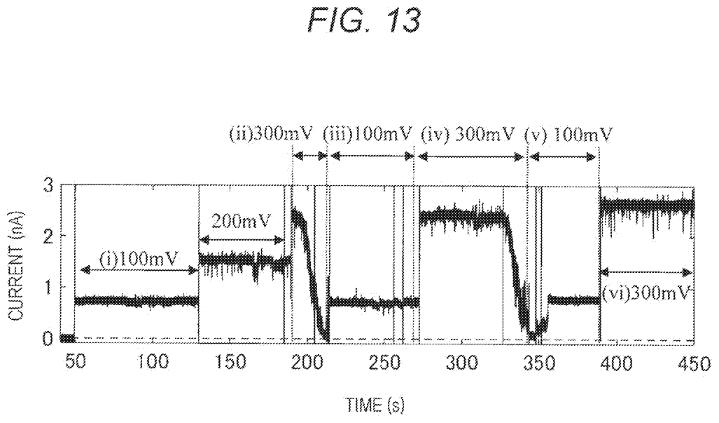

FIG. 13 is a diagram showing a current waveform when MBD2 is measured in Example 5. As shown in FIG. 13, a voltage of 100 mV was applied in a section (i); a voltage of 200 mV was applied between the section (i) and a section (ii); a voltage of 300 mV was applied in the section (ii); a voltage of 100 mV was applied in a section (iii); a voltage of 300 mV was applied in a section (iv); a voltage of 100 mV was applied in a section (v); and a voltage of 300 mV was applied in a section (vi).

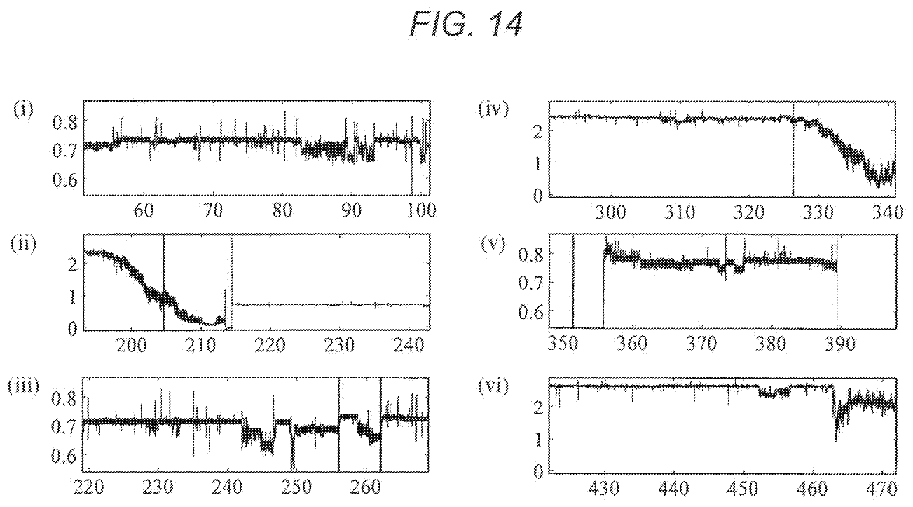

FIG. 14 is a diagram showing current waveforms obtained by enlarging sections (i) to (vi) in FIG. 13. As shown in FIG. 14, it can be seen that a current value gradually decreases in the sections (ii) and (iv) where a voltage of 300 mV is applied. This is considered to be because a large amount of MBD2 is drawn to the nanopore and the nanopore is clogged. Here, in the sections (iii) and (v), when an applied voltage was changed to 100 mV, the current value was recovered. It was confirmed that the passing current waveform of the protein can be obtained as in before the clogging.

### Comparative Example 4: Measurement of Protein Using Nanopore without Coating

As in Comparative Example 1, a nanopore device without coating was produced. Thereafter, the same measurement solution as that in Example 5 was used. A voltage was applied in the range of 100 to 200 mV between electrodes, and an ion current was measured.

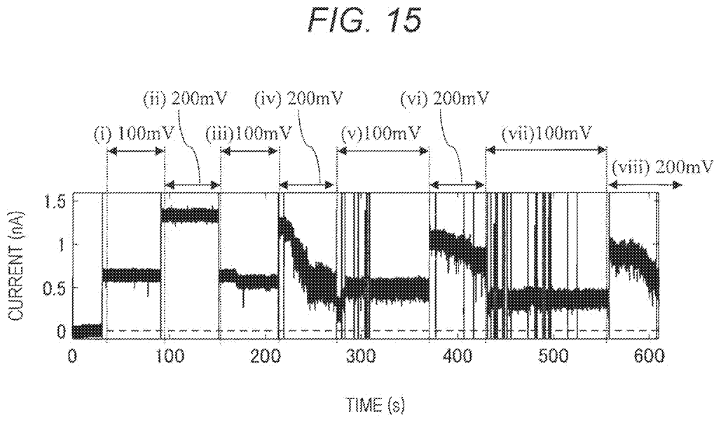

FIG. 15 is a diagram showing a current waveform when MBD2 is measured in Comparative Example 4. As shown in FIG. 15, a voltage of 100 mV was applied in a section (i); a voltage of 200 mV was applied in a section (ii); a voltage of 100 mV was applied in a section (iii); a voltage of 200 mV was applied in a section (iv); a voltage of 100 mV was applied in a section (v); a voltage of 200 mV was applied in a section (vi); a voltage of 100 mV was applied in a section (vii); and a voltage of 200 mV was applied in a section (viii).

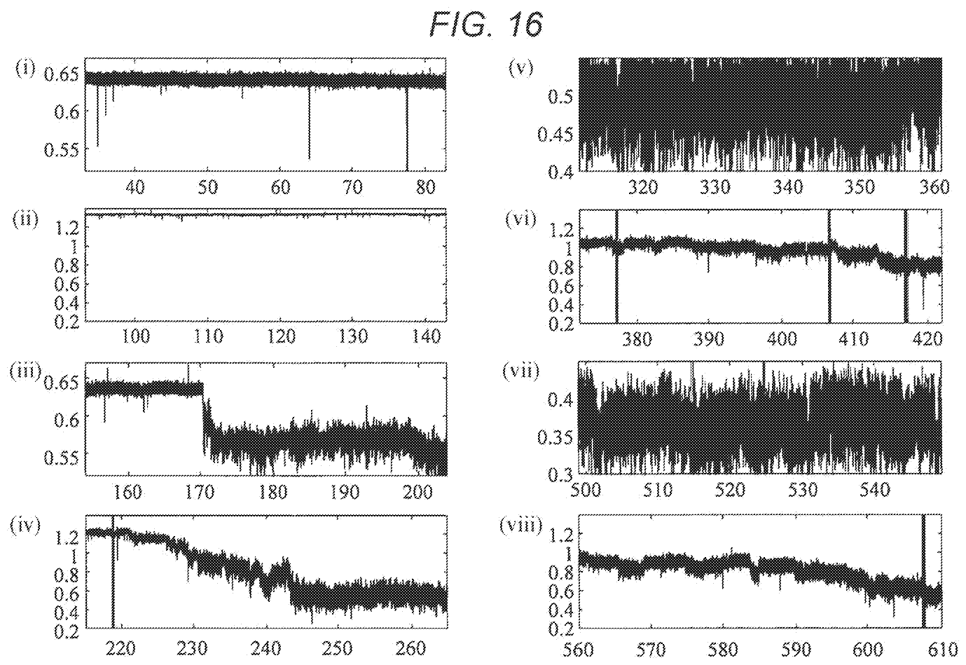

FIG. 16 is a diagram showing current waveforms obtained by enlarging sections (i) to (viii) in FIG. 15. As shown in FIG. 16, when a voltage of 200 mV was applied in the sections (iv), (vi), and (viii), a decrease in the current value, which was considered to be derived from the clogging of the nanopore, was observed. Therefore, an attempt was made to eliminate the clogging by applying a pulse voltage after returning the voltage to 100 mV (section (iii) and section (iv)). As a result, although the current value recovered to the level before the clogging, the passing current waveform of the protein was buried in noise, and could not be acquired. This is considered to be because irreversible adsorption occurred when MBD2 was drawn to the nanopore by applying a voltage of 200 mV.

From the results of Example 5 and Comparative Example 4, it could be confirmed that the nanopore coating method of the present disclosure can exhibit the effect of suppressing adsorption even to the positively charged object to be measured (protein).

### MODIFIED EXAMPLES

The present disclosure is not limited to the above-described embodiments, and includes various modified examples. For example, the above-described embodiments have been described in detail to clearly describe the present disclosure, and the present invention need not necessarily include all the described configurations. A part of one embodiment can be replaced by the configuration of another embodiment. A configuration of another embodiment can also be added to the configuration of one embodiment. It is also possible to, for a part of the configuration of each embodiment, add, delete, or replace a part of the configuration of another embodiment.

What is claimed is:

1. A nanopore forming method comprising:
disposing a membrane between a first electrolyte solution and a second electrolyte solution; and
bringing a first electrode into contact with the first electrolyte solution and a second electrode into contact with the second electrolyte solution;
applying a first voltage between the first electrode and the second electrode to form a nanopore in the membrane,
wherein at least one of the first electrolyte solution or the second electrolyte solution contains a first substance that is an organic substance physically adsorbed or chemically adsorbed to the membrane to form a molecular layer.

2. The nanopore forming method according to claim 1, wherein the first substance is a substance having a structure capable of interacting with a surface of the nanopore to be adsorbed to the surface.

3. The nanopore forming method according to claim 1, wherein the first substance has a hydrophilic structure.

4. The nanopore forming method according to claim 1, wherein the first substance has a hydrophobic moiety at one end portion and a hydrophilic moiety at another end portion in a molecular structure, or is an amphiphilic molecule having a hydrophobic moiety located at one portion and a hydrophilic moiety located at another portion when having a three-dimensional structure.

5. The nanopore forming method according to claim 1, wherein the first substance is a surfactant.

6. The nanopore forming method according to claim 5, wherein the surfactant is at least one selected from the group consisting of polyoxyethylene sorbitan fatty acid ester, sulfate ester, polyoxyethylene alkyl ether, alkyltrimethylammonium bromide, polyoxyethylene alkylphenyl ether, cholate, sarcosyl, alkylpolyglycoside DDM, digitonin, 3-[(3-cholamidopropyl)dimethylammonio]-1-propanesulfonate, and 3-[(3-cholamidopropyl)dimethylammonio]-2-hydroxy-1-propanesulfonate.

7. The nanopore forming method according to claim 1, wherein a concentration of the first substance is 1 μM or more or 0.0001% by weight or more.

8. The nanopore forming method according to claim 1, wherein a material of the membrane is HfO₂, SiO₂, TiO₂, SiN, SiON, SiC, SiCN, Al₂O₃, HfAlOₓ, ZrAlOₓ, TaO₃, graphene, a carbon membrane, or a composite material containing these materials.

9. The nanopore forming method according to claim 1, further comprising:
replacing the first electrolyte solution and the second electrolyte solution by a third electrolyte solution containing the first substance after formation of the nanopore; and
immersing the nanopore in the third electrolyte solution for a predetermined time.

10. The nanopore forming method according to claim 9, wherein a pH of the third electrolyte solution is different from a pH of the first electrolyte solution and a pH of the second electrolyte solution.

11. The nanopore forming method according to claim 1, further comprising replacing the first electrolyte solution and the second electrolyte solution by a solution not containing the first substance after formation of the nanopore.

12. A nanopore measuring method comprising:
performing the nanopore forming method according to claim 1; and
replacing at least one of the first electrolyte solution or the second electrolyte solution by a measurement solution containing an object to be measured after formation of the nanopore.

13. The nanopore measuring method according to claim 12, further comprising applying a second voltage between the first electrode and the second electrode after the replacement to measure a change in a current signal flowing through the nanopore.

14. The nanopore measuring method according to claim 12, wherein the object to be measured is a string-like substance that can be dispersed or dissolved in the measurement solution and has a diameter of about 0.1 nm to 500 nm, or has a diameter of 0.1 nm to 500 nm when approximated to a sphere.

15. The nanopore measuring method according to claim 12, wherein the object to be measured contains a molecule having a biopolymer, a biomonomer, or a derivative thereof in a structure thereof.

16. The nanopore measuring method according to claim 12, wherein the object to be measured contains at least one selected from the group consisting of a nanoparticle, nanostructure, or complex with a biomolecule, of an inorganic substance, metal, or organic substance.

17. The nanopore measuring method according to claim 12, wherein the object to be measured contains at least one selected from the group consisting of a cell, an organelle, and a virus.
